(19) United States
(12) Patent Application Publication
(10) Pub. No.: US 2010/0041630 A1
(43) Pub. Date: Feb. 18, 2010

(54) Methods for Treating or Preventing Inflammation Using a Glycerophosphate Salt

(75) Inventor: Alan E. Kligerman, Egg Harbor Township, NJ (US)

Correspondence Address: PANITCH SCHWARZE BELISARIO & NADEL LLP, ONE COMMERCE SQUARE, 2005 MARKET STREET, SUITE 2200, PHILADELPHIA, PA, 19103, US

(73) Assignee: PRELIEF INC., Pleasantville, NJ (US)

(21) Appl. No.: 12/526,818

(22) PCT Filed: Feb. 13, 2008
(86) PCT No.: PCT/US08/53790

§ 371 Date: Aug. 12, 2009

Related U.S. Application Data
(60) Provisional application No. 60/889,765, filed on Feb. 14, 2007.

(51) Int. Cl. A61K 31/66

(52) U.S. Cl. 514/129

(57) ABSTRACT

Glycerophosphate salts have been found to mitigate the syndromes or conditions of discomfort associated with inflammation. Therefore, methods are provided for treating or preventing an inflammatory condition using a glycerophosphate salt. In particular, methods are provided for treating or preventing an inflammatory condition using calcium glycerophosphate (CGP).

### BACKGROUND OF THE INVENTION

Inflammation is a complex, localized, protective process initiated by tissue injury or destruction, which serves to destroy, dilute, or sequester both the injurious agent and the injured tissue. Histologically, inflammation involves a complex series of events, including dilatation of arterioles, capillaries and venules, with increased permeability and blood flow, exudation of fluids, including plasma proteins and leucocytic migration into the inflammatory focus.

Although inflammation has evolved as a protective response against injury and infection by a physical, chemical, or biologic agent, in certain cases inflammation itself can cause much of the physical discomfort, e.g., pain and loss of function, that has come to be associated with different diseases and injuries. Accordingly, it is a common medical practice to administer pharmacological agents which have the effect of neutralizing the inflammatory response in these cases. Agents having these properties are classified as anti-inflammatory drugs. Anti-inflammatory drugs are used for the treatment of a wide spectrum of disorders, and the same drugs are often used to treat different diseases or disorders.

The anti-inflammatory drugs are a heterogeneous group of compounds, often chemically unrelated, which nevertheless share certain therapeutic actions and side-effects. Examples of anti-inflammatory drugs include corticosteroids, which are hormones, specifically glucocorticoids, that reduce inflammation by binding to cortisol receptors. Another class of anti-inflammatory drugs is non-steroidal anti-inflammatory drugs (NSAIDs). It is believed that NSAIDs work by inhibiting the production of prostaglandins (PGs), a group of compounds derived from unsaturated 20-carbon fatty acids, primarily arachidonic acid, via the cyclooxygenase (COX) pathway. Examples of NSAIDs include, but are not limited to, aspirin, ibuprofen, ketoprofen, piroxicam, naproxen, sulindac, choline subsalicylate, diflunisal, fenoprofen, indomethacin, meclofenamate, salsalate, tolmetin, and magnesium salicylate. Additional examples of steroidal and non-steroidal anti-inflammatory agents are listed in the Physicians Desk Reference, 54 Edition, (2000) (see pp. 202 and 217 for an index of such preparations).

The natural and synthetic corticosteroid preparations cause a number of adverse drug reactions (ADRs), including elevation of blood pressure, salt and water retention, and increased potassium and calcium excretion. Moreover, corticosteroids may mask the signs of infection and enhance dissemination of infectious microorganisms. These hormones are not considered safe for use in pregnant females, and long-term corticosteroid treatment has been associated with gastric hyperactivity and/or peptic ulcers. Treatment with these compounds may also aggravate diabetes mellitus, requiring higher doses of insulin, and may produce psychotic disorders. Hormonal anti-inflammatory agents which indirectly increase the production of endogenous corticosteroids have the same potential for adverse side-effects.

NSAIDS are also associated with a wide spectrum of undesirable side-effects. For example, use of NSAIDs can cause direct and indirect irritation of the gastrointestinal tract (GIT), resulting in ADRs, such as nausea, dyspepsia, gastric ulceration/bleeding, and diarrhea. Risk of ulceration increases with long duration and with higher doses of NSAIDs. NSAIDs are also associated with a relatively high incidence of renal ADRs, such as salt and fluid retention and hypertension, and in rare instances, more severe renal conditions, such as interstitial nephritis, nephrotic syndrome, acute renal failure, and acute tubular necrosis. A recent meta-analysis of trials comparing NSAIDs found that, with the exception of naproxen, both the selective Cox-2 inhibitor and the traditional NSAID are associated with an increased cardiovascular risk (Kearney et al, BMJ 332:1302-1308 (2006)).

Therefore, there is a continuing need to develop novel therapy that reduces inflammation without having deleterious side effects. Moreover, such therapeutic agent should be simple and relatively inexpensive to prepare, its activity should be reproducible among preparations and remain stable over time, and its anti-inflammatory effects should be achievable with dose regimens that are associated with minimal toxicity.

### BRIEF SUMMARY OF THE INVENTION

It is now discovered that a glycerophosphate salt can be used to treat or inhibit the onset of an inflammatory condition.

In one general aspect, an embodiment of the present invention comprises a method of treating an inflammatory condition in a subject comprising administering to the subject a therapeutically effective amount of a glycerophosphate salt, to interdict, prevent, palliate, or alleviate the inflammatory condition in the subject, provided that the inflammatory condition is associated with one or more diseases other than those selected from the group consisting of irritable bowel syndrome, interstitial cystitis, hemorrhagic cystitis, inflammatory bowel disease, and fibromyalgia.

In another general aspect, an embodiment of the present invention comprises a method of preventing or inhibiting the onset of an inflammatory condition in a subject comprising administering to the subject a prophylatically effective amount of a glycerophosphate salt, to interdict, prevent, palliate, or delay the onset of the inflammatory condition in the subject, provided that the inflammatory condition is associated with one or more diseases other than those selected from the group consisting of irritable bowel syndrome, interstitial cystitis, hemorrhagic cystitis, inflammatory bowel disease, and fibromyalgia.

Other aspects, features and advantages of the invention will be apparent from the following disclosure, including the detailed description of the invention and its preferred embodiments and the appended claims.

### DETAILED DESCRIPTION OF THE INVENTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the present invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. In this application, certain terms are used frequently, which shall have the meanings as set in the specification. It must be noted that as used herein and in the appended claims, the singular forms “a,” “an,” and “the” include plural reference unless the context clearly dictates otherwise.

Embodiments of the present invention comprise methods of inhibiting the onset of or treating an inflammatory condition in a subject comprising administering to the subject a prophylatically or therapeutically effective amount, respectively, of a glycerophosphate salt, thereby interdicting, preventing, delaying, palliating, or alleviating the inflammatory condition, provided that the inflammatory condition is associated with one or more diseases other than those selected from the group consisting of irritable bowel syndrome, interstitial cystitis, hemorrhagic cystitis, inflammatory bowel disease, and fibromyalgia.

As used herein the term “inflammation” is intended to encompass both acute responses (i.e., a response in which the inflammatory processes are active) and chronic responses (i.e., a response marked by slow progress and formation of new connective tissue). Chronic and acute inflammation may be distinguished by the cell types involved. Acute inflammation often involves polymorphonuclear neutrophils; whereas chronic inflammation is normally characterized by a lymphohistiocytic and/or granulomatous response. Examples of specific types of inflammation are diffuse inflammation, focal inflammation, croupous inflammation, interstitial inflammation, obliterative inflammation, reactive inflammation, specific inflammation, toxic inflammation, and traumatic inflammation. The term “inflammation” encompasses both internal inflammation and external inflammation.

As used herein, the term “inflammatory condition” refers to a symptom or manifestation associated with inflammation. In particular embodiments, an inflammatory condition is manifested by one or more symptoms selected from the group consisting of redness (rubor), heat (calor), swelling (tumor), pain (dolor) and dysfunction of the tissues or organs involved (functio laesa). All of the five conditions may be observed or associated with an inflammation, but none is necessarily always present. The degree or the severity of the five conditions may vary. For example, the heat, can be localized to the site of inflammation, or it can be systemic, with various degree of elevation of the body temperature. The dysfunction of the tissues or organs involved can be a partial or a total failure of the tissue or organ. It can range from annoying to life-threatening, depending on severity and on the type of tissue or organ in which the dysfunction occurs.

As used herein, the “swelling” refers to the enlargement of any organ or tissue due to accumulation of excessive fluid in the organ or tissue. In particular embodiments, the swelling is edema, which refers to the accumulation of an excessive amount of watery fluid in cells or intercellular tissues. Edema can accumulate excessive liquid in almost any location in the body, with the most common sites at the feet and ankles. It can be localized, for example, due to venous or lymphatic obstruction or increased vascular permeability. It can also be systemic, for example, due to heart failure or renal disease.

As used herein, “pain” refers to an unpleasant sensation associated with actual or potential tissue damage and mediated by specific nerve fibers to the brain where its conscious appreciation may be modified by various factors. The unpleasant sensation of pain ranges in intensity from slight through severe to indescribable. Pain is experienced as having qualities such as stinging, sharp, throbbing, dull, nauseating, burning and shooting.

An inflammatory condition can be associated with any disease or disorder in which inflammation occurs, provided that the disease or disorder is not selected from the group consisting of irritable bowel syndrome, interstitial cystitis, hemorrhagic cystitis, inflammatory bowel disease, fibromyalgia, and combinations thereof. In some embodiments of the invention, the inflammatory condition is associated with a disease or a condition selected from the group consisting of rheumatoid arthritis, osteoarthritis, arthropathy, ankylosing spondylitis, psoriatic arthritis, reactive arthritis, gout, dysmenorrhoea, metastatic bone pain, headache, migraine, postoperative pain, mild-to-moderate pain due to inflammation and tissue injury, renal colic, appendicitis, gastritis, otitis, pancreatitis, laryngitis, asthma, chronic pelvic pain syndrome (CPPS), chronic abacterial prostatitis, insult, trauma, an allergic response, an allergic reaction, a condition involving a mast cell mediator and combinations thereof. Examples of the arthropathy include, but are not limited to, reactive arthropathy, enteropathic arthropathy, crystal arthropathy, diabetic arthropathy, neuropathic arthropathy, and combinations thereof. Examples of mast cell mediator include, but are not limited to, histamine, cytokines, and proteolytic enzymes. The inflammatory condition can be associated with, for example, brain inflammation, meningeal inflammation, skin inflammation, inflammatory arthritis, cardiopulmonary inflammation, bladder inflammation, and gastrointestinal inflammation. Method of the present invention can be used to treat or prevent any inflammatory conditions.

In one embodiment of the present invention, the inflammatory condition is mucositis, inflammation of a mucous membrane, including, but not limited to, the mucous membranes lining the mouth and digestive tract. Mucositis usually occurs as an adverse effect of chemotherapy and radiotherapy treatment for cancer. For example, oral and gastrointestinal (GI) mucositis can affect up to 100% of patients undergoing high-dose chemotherapy and hematopoietic stem cell transplantation, 80% of patients with malignancies of the head and neck receiving radiotherapy, and a wide range of patients receiving chemotherapy. Method of the present invention can be used to treat or prevent mucositis. In a preferred embodiment of the present invention, an effective amount of a glycerophosphate salt is administered topically to the mucous membrane, thereby interdicting, preventing, delaying the onset of, palliating, or alleviating the inflammatory condition associated with mucositis.

In yet another embodiment of the present invention, the inflammatory condition is associated with vulvar vestibulitis, inflammation of the vulva in vestibular region. The inflammatory condition can be pain, such as burning pain or other types of pain, a surface irritation, redness of the surface, etc. In a preferred embodiment of the present invention, an effective amount of a glycerophosphate salt is administered topically to the vestibular region of the vulva, thereby interdicting, preventing, delaying the onset of, palliating, or alleviating the inflammatory condition associated with vulvar vestibulitis.

As used herein, the term “subject” refers to an animal, preferably a mammal, most preferably a human, who has been the object of treatment, observation or experiment. Examples of a subject can be a human, a livestock animal (beef and dairy cattle, sheep, poultry, etc.), a companion animal (dog, cat, horse, etc), or a race animal, e.g., a race horse.

As used herein the term “treatment”, “treat” or “therapy” refers to the prevention of deterioration of a disease, disorder or condition when a patient contracts such a disease, disorder or condition, preferably, at least maintenance of the status quo, and more preferably, alleviation, still more preferably, resolution of the disease, disorder or condition.

As used herein the term “prophylaxis”, “prevent” or “prevention” refers to, when referring to a disease, disorder or condition, a type of treatment conducted before such a disease, disorder or condition occurs such that the disease, disorder or condition will not occur, will be delayed to occur, or will occur but will deteriorate to a less degree.

As used herein, the term “treat” or “prevent” in the broadest sense, with respect to a disease, disorder or condition, refers to any medical act thereto, and include any act for diagnosis, therapy, prevention, prognosis and the like.

When used for treating or preventing an inflammatory condition, a glycerophosphate salt can be used as a reliever which is used after the onset of the inflammation. The glycerophosphate salt can also be used as a controller which is used for long-term control to prevent the occurrence of the inflammation. Those skilled in the art will be able to use an effective amount of a glycerophosphate salt for either therapy or prevention of the inflammatory condition.

The term “effective amount” as used herein, means that amount of a glycerophosphate salt that elicits the biological or medicinal response in a tissue system of a subject, or in a subject, that is being sought by a researcher, veterinarian, medical doctor or other clinician, which includes interdicting, preventing, palliating, or alleviating a syndrome or a condition of discomfort of the subject associated with the inflammatory condition being treated. The administration of an effective amount of a glycerophosphate salt to a subject results in a clinically observable beneficial effect. The clinically observable beneficial effect can be a situation that an observable inflammatory condition is prevented from further development or aggravation or develop to a lesser degree, than without administration of the composition of the present invention. The clinically observable beneficial effect can also be a situation that inflammatory condition is prevented from occurring or subsequently occurs to a lesser degree than without administration of the composition of the present invention, when the composition is administered to a subject before the inflammatory condition is observable. The effective amount can be a therapeutically effective amount or a prophylactically effective amount.

Methods are known in the art for determining therapeutically and prophylactically effective doses of a glycerophosphate salt according to disclosure of the present invention. A useful assay for confirming an effective amount (e.g., a therapeutically effective amount) for a predetermined application is to measure the degree of recovery from a target disease. An amount actually administered depends on an individual to be treated. The amount is preferably optimized so as to obtain a desired effect without significant side effects. The determination of a prophylactically or therapeutically effective amount is within the ability of those skilled in the art in view of the present disclosure. A prophylactically or therapeutically effective amount of any compound can be estimated using either a cell culture assay or any appropriate animal model. The animal model is used to achieve a desired concentration range and an administration route. Thereafter, such information can be used to determine a dose and route useful for administration into humans.

The therapeutic effect and toxicity of a compound may be determined by standard pharmaceutical procedures in cell cultures or experimental animals (e.g., ED₅₀, a dose therapeutically effective for 50% of a population; and LD₅₀, a dose lethal to 50% of a population). The dose ratio between therapeutic and toxic effects is a therapeutic index, and it can be expressed as the ratio of ED₅₀/LD₅₀. Pharmaceutical compositions which exhibit high therapeutic indices are preferable. The data obtained from cell culture assays and animal studies can be used for formulating a dosage range for use in humans. The dosage of such compounds lies preferably within a range of circulating concentrations that include the ED₅₀, with little or no toxicity. Such a dosage may vary within this range depending upon the dosage form employed, the susceptibility of a patient, and the route of administration. Guidance for specific doses and delivery methods is provided in publications known in the art. Procedures can be performed to evaluate the effect of the administration of a glycerophosphate salt to a subject, thus allowing a skilled artisan to determine the effective amount of a glycerophosphate salt to be administered to the subject. For example, the redness or the swelling of the tissue or organ can be measured by visual inspection prior to or after the administration of the glycerophosphate salt. The heat can be measured by a thermometer.

Techniques, such as surveys or animal models, can be used to evaluate the effectiveness of a glycerophosphate salt in treating or preventing an inflammatory condition. Various animal models of pain or inflammation exist, for example, the neuroinflammation models (Chacur et al., Pain 94: 231-44 (2001); Milligan et al., Brain Res 861: 105-16 (2000)), acute nociceptive test models and inflammatory models (Brennan, T. J. et al. Pain 64:493 (1996); D'Amour, F. E. and Smith, D. L. J Pharmacol 72: 74-79 (1941); Eddy, N. B. et al. J Pharmacol Exp Ther 98:121 (1950); Hafffer, F. Dtsch Med Wochenschr 55:731 (1929); Hargreaves, K. et al. Pain 32: 77-88 (1988); Hunskaar, S. et al. J Neurosci Meth 14:69 (1985); Randall, L. O. and Selitto, J. J. Arch. Int. Pharmacodyn 111: 409-419 (1957); Siegmund, E. et al. Proc Soc Exp Bio Med 95:729 (1957)). In addition, a suitable animal model for evaluating anti-inflammatory activity is described by Whitehouse et al. Inflammopharmacology 7:89-105 (1999).

The exact dose is chosen by an individual physician in view of the condition of a patient to be treated. Doses and administration are adjusted to provide a sufficient level of the active portion, or to attain a desired effect. Preferably, a therapeutically effective amount of a glycerophosphate salt will reduce a syndrome or a condition of discomfort of the subject associated with an inflammatory condition under treatment by at least about 20%, for example, by at least about 30%, about 40%, about 50%, about 60%, about 70%, about 80%, about 90%, or about 100%. Preferably, a prophylactically effective amount of a glycerophosphate salt will reduce a syndrome or a condition of discomfort of the subject associated with an inflammatory condition to be prevented, or the probability of its onset, by at least about 20%, for example, by at least about 30%, about 40%, about 50%, about 60%, about 70%, about 80%, about 90%, or about 100%.

The therapeutically effective amount or the prophylactically effective amount of a glycerophosphate salt per administration can be, for example, about 0.1 gram to about 3.0 grams, and preferably about 0.3 gram to about 1.0 gram. The preferred daily dosage of a glycerophosphate salt can be about 0.6 gram to about 18 grams, and more preferably about 1.8 grams to about 6 grams. However, the number of doses per day and the quantity of the glycerophosphate salt which may be administered to a subject can be virtually unlimited.

As used herein, the term “glycerophosphate salt” refers to a chemical compound that is derived from glycerophosphate, in which one or more of the hydrogens of the phosphate group of glycerophosphate are replaced by a basic radical, in particular embodiments by a metal ion. As used herein, the term “glycerophosphate” refers to an anion of a phosphoric ester of glycerol, in which a carbon atom of glycerol bonds to an oxygen atom in the phosphate group of phosphoric acid. A glycerophosphate salt can be a chiral molecule, i.e., it can exist in two forms that are nonsuperimposable mirror images. It is intended that the present invention includes within its scope the stereochemically pure isomeric forms of a glycerophosphate salt and/or their racemates.

In particular embodiments, methods of the invention utilize one or more glycerophosphate salts selected from the group consisting of calcium glycerophosphate (CGP), magnesium glycerophosphate, zinc glycerophosphate, manganese glycerophosphate, lithium glycerophosphate, cupric glycerophosphate, ferric glycerophosphate, quinine glycerophosphate, glycerophosphate disodium, glycerophosphate dipotassium, glycerophosphate barium, and glycerophosphate strontium.

In a preferred embodiment, methods of the invention utilize calcium glycerophosphate. As used herein, the term “calcium glycerophosphate” or “CGP,” also known as “glycerophosphate calcium,” refers to a chemical compound having a molecular formula of C₃H₇CaO₆P in its anhydrous form. “CGP” can also exist as a hydrate, including the monohydrate and the dihydrate. Examples of calcium glycerophosphate include, but are not limited to, any one, or any combination of two or more of the three isomers of CGP, namely β-glycerophosphoric acid calcium salt ((HOCH₂)₂CHOPO₃Ca) and D(+) and L(−)-α-glycerophosphoric acid calcium salt (HOCH₂CH(OH)CH₂OPO₃Ca).

Calcium glycerophosphate available from various commercial sources can be used in the present invention. In one embodiment, Prelief®, a dietary supplement for use in reducing the impact of acid in foods and beverages that is available from AkPharma Inc. (Pleasantville, N.J. 08232), in either tablet or powder form, can be used in the present invention for orally administering the calcium glycerophosphate to the subject. Other commercially available CGP also includes CGP available from Astha Laboratories Pvt, Ltd, B-4, Industrial Estate, Sanathnagar, Hyderabad-18, India, and Seppic Inc., 30 Two Bridges Road, Fairfield, N.J.

In particular embodiments, one or more glycerophosphate salts are combined with an acceptable carrier in a composition for administration to a subject. The composition can contain about 0.1 mg to about 3000 mg of a glycerophosphate salt, and can be constituted into any form suitable for the mode of administration selected. Carriers include one or more excipients, including, but not limited to, binders, suspending agents, lubricants, flavorants, sweeteners, preservatives, dyes, and coatings.

To prepare the compositions, one or more glycerophosphate salts, as the active ingredient, are intimately admixed with a carrier according to conventional pharmaceutical compounding techniques, which carrier may take a wide variety of forms depending on the form of preparation desired for administration. Thus, if the composition is intended to be administered intranasally or by inhalation, for example, the composition may be a converted to a powder or aerosol form, as conventional in the art, for such purposes. Other formulations, such as for oral or parenteral delivery, are also used as conventional in the art. In preparing the compositions in oral dosage form, any of the usual pharmaceutical media may be employed. For liquid oral preparations, for example, suspensions, elixirs and solutions, suitable carriers and additives include water, glycols, oils, alcohols, flavoring agents, preservatives, coloring agents and the like; for solid oral preparations, for example, powders, capsules, caplets, gelcaps and tablets, suitable carriers and additives include starches, sugars, diluents, granulating agents, lubricants, binders, disintegrating agents and the like.

Compositions for administration herein may form solutions, suspensions, tablets, pills, capsules, spray, drops, sustained release formulations or powders. Because of their ease in administration, tablets and capsules represent the most advantageous oral dosage unit form, in which case solid pharmaceutical carriers are obviously employed. If desired, tablets may be sugar coated or enteric coated by standard techniques. For parenterals, the carrier will usually comprise sterile water, though other ingredients, for example, for purposes such as aiding solubility or for preservation, may be included. Injectable suspensions may also be prepared, in which case appropriate liquid carriers, suspending agents and the like may be employed. The compositions herein can contain, per dosage unit, e.g., tablet, capsule, powder, injection, teaspoonful and the like, an amount of the active ingredient necessary to deliver an effective amount as described above.

A glycerophosphate salt can be administered to a subject by one or more routes of administration selected from the group consisting of nasal administration; oral administration; inhalation administration; subcutaneous administration; transdermal administration; intravenous administration; topical administration; buccal administration; intraperitoneal administration; intraocular administration; peri-ocular administration; intramuscular administration; implantation administration; infusion, intravesical instillation; and central venous administration. Preferably, a glycerophosphate salt is administered orally. It can be administered with foods, beverages or additional one or more other drugs. It can be orally administered in solid forms, such as pills, tablets, caplets, capsules (each including immediate release, timed release and sustained release formulations), granules, and powders, and/or in liquid forms, such as solutions, syrups, elixirs, emulsions, and suspensions.

A glycerophosphate salt can also be administered by injection or infusion into a vein (intravenous, IV), a muscle (intramuscular, IM), or under the skin (subcutaneous, SC). It can be injected or infused in the form of sterile solutions, emulsions or suspensions. A glycerophosphate salt administered by IM is absorbed into the blood more slowly than IV, thus may have longer lasting effect than IV. A glycerophosphate salt can also be given by a catheter or port permanently inserted into a central vein or body cavity. A port is a small reservoir or container that is placed in a vein or under the skin in the area where the drug will be given. These methods eliminate the need for repeated injections. Catheters and ports can also be placed in the bladder or pelvis.

A glycerophosphate salt can be administered directly to a bladder or rectum by intravesical infusion or intravesical instillation using local enema or a suppository.

A glycerophosphate salt can also be given external topically as a cream or ointment applied directly to the surface areas exposed to or affected by the inflammation, such as the mucous membrane for mucositis or the vulvar surface for vulvar vestibulitis. Also, a glycerophosphate salt can be administered via transdermal skin patches to provide continuous dosage regimen. A glycerophosphate salt can also be applied internal topically, for example, into the rectum, vagina, urine tract, and/or colon by a procedure such as enema, lavage, or gavage. A glycerophosphate salt can further be administered by nasal application, for example, via spray or dropper. A glycerophosphate salt can also be administered by intra-ocular or peri-ocular application.

How often and how long a glycerophosphate salt is administered to a subject depends on the type of treatment or prevention, how the subject responds to the glycerophosphate salt, factors associated with the subject, e.g., age, weight, diet, health, etc., ability to tolerate the glycerophosphate salt, and the types of glycerophosphate salts used. A glycerophosphate salt can be administered on a regimen of one to multiple times per day. Alternatively, a glycerophosphate salt can be formulated in an extended release form suitable for once-weekly or once-monthly administration. Methods are known to those skilled in the art to manufacture the extended release dosage form. Preferably, a glycerophosphate salt is administered to the subject at intervals during the day, such as at breakfast, lunch, dinner, and upon retiring.

A glycerophosphate salt can be given in combination with one or more other drugs. They can be administered simultaneously or one following the other. In particular embodiments, a glycerophosphate salt can be administered in combination with one or more additional anti-inflammation agents. The additional anti-inflammation agents are selected from the group consisting of: (1) serotonin receptor antagonists; (2) serotonin receptor agonists; (3) histamine receptor antagonists; (4) bradykinin receptor antagonists; (5) kallikrein inhibitors; (6) tachykinin receptor antagonists, including neurokinin₁ and neurokinin₂ receptor subtype antagonists; (7) calcitonin gene-related peptide (CGRP) receptor antagonists; (8) interleukin receptor antagonists; (9) inhibitors of enzymes active in the synthetic pathway for arachidonic acid metabolites, including (a) phospholipase inhibitors, including PLA₂ isoform inhibitors and PLC_{γ} isoform inhibitors (b) cyclooxygenase inhibitors, and (c) lipooxygenase inhibitors; (10) prostanoid receptor antagonists including eicosanoid EP-1 and EP-4 receptor subtype antagonists and thromboxane receptor subtype antagonists; (11) leukotriene receptor antagonists including leukotriene B₄ receptor subtype antagonists and leukotriene D₄ receptor subtype antagonists; (12) opioid receptor agonists, including mu-opioid, delta-opioid, and kappa-opioid receptor subtype agonists; (13) purinoceptor agonists and antagonists including P_{2X} receptor antagonists and P_{2Y} receptor agonists; (14) adenosine triphosphate (ATP)-sensitive potassium channel openers. In other embodiments, a glycerophosphate salt can be administered in combination with one or more anti-histamines. In yet other embodiments, a glycerophosphate salt can be administered in combination with pentosan polysulfate (PPS) or other heparin-like macromolecular carbohydrate derivatives.

In other particular embodiments, a glycerophosphate salt can be administered in combination with one or more additional anti-inflammation agents that also act as anti-spasm agents. Suitable anti-inflammatory agents that also act as anti-spasm agents include serotonin receptor antagonists, tachykinin receptor antagonists, ATP-sensitive potassium channel openers and calcium channel antagonists. Other agents that may be utilized in the combination specifically for their anti-spasm properties including endothelin receptor antagonists, calcium channel antagonists and the nitric oxide donors (enzyme activators).

While not wishing to be bound by theory, a glycerophosphate salt can be used to treat or prevent an inflammatory condition at least in part due to its acid-binding capability to neutralize the naturally-created acid in some wounds. Many wounds, especially burns of a certain level and lacerations that present a bloody and at least partial skin-denuded area of at least the first epidermal level, become acidotic, probably providing a body defense mechanism against microbial infections. Such acid may be body-protective but is also painful, as it stings any exposed nerves. Preferably, a glycerophosphate salt is applied to the wound either after the application of or simultaneously with an anti-infective agent, such as an antibiotic agent, or a bactericide, e.g., povidone, alcohol, etc.

Another possible mechanism for the anti-inflammation function of a glycerophosphate salt may be that the quicker repair and replacement of cells occasioned by the presence of the glycerophosphate salt. Various observations suggest that a glycerophosphate salt functions to promote epidermal cell renewal, see for example, US20040037766. The quick repair and replacement of epidermal cells provide, among other things, enhanced ceramide synthesis, which hastens repair of the skin's surface and provides tighter cell-to-cell adhesion, preventing invasion between vulnerable cell walls of irritating substances.

This invention will be better understood by reference to the non-limiting examples that follow, but those skilled in the art will readily appreciate that the examples are only illustrative of the invention as described more fully in the claims which follow thereafter.

### EXAMPLE 1

### Determining the Therapeutic Effectiveness of Calcium Glycerophosphate in an Animal Model

This example depicts a method of determining the therapeutic effectiveness of calcium glycerophosphate (CGP) in treating an inflammatory condition in rats. Methods similar to that exemplified herein, however, are equally applicable to determining the therapeutic effectiveness of any glycerophosphate salt in treating any inflammatory condition in any animal.

Rats with inflammatory condition associated with polyarthritis are generated by injecting about 300-1000 ug of heat-killed Mycobacterium tuberculosis suspended in squalene into the tail vein of rats. Various amounts of CGP, e.g., about 0.1 gram to about 3.0 grams per administration, a control anti-inflammatory agent, e.g., 300 mg/kg of aspirin, or a placebo is administered to the inflammatory rats via injection 1 to 5 times a day for 3-10 days. Activity of disease is assessed by measuring the swelling of all four paws of the rats with a micrometer screw gauge. An overall arthritis score is independently assessed. The activity of the various amounts of CGP is compared to that of the control anti-inflammatory agent and that of the placebo.

A therapeutically effective amount of CGP will reduce the overall arthritis score by at least about 20%, for example, by at least about 30%, about 40%, about 50%, about 60%, about 70%, about 80%, about 90%, or about 100%.

### EXAMPLE 2

### Determining the Prophylactic Effectiveness of Calcium Glycerophosphate in an Animal Model

This example depicts a method of determining the prophylactic effectiveness of calcium glycerophosphate (CGP) in preventing an inflammatory condition in rats. Methods similar to that exemplified herein, however, are equally applicable to determining the prophylactic effectiveness of any glycerophosphate salt in preventing any inflammatory condition in any animal.

Various amounts of CGP, e.g., about 0.1 gram to about 3.0 grams per administration, a control anti-inflammatory agent, e.g., 300 mg/kg of aspirin, or a placebo is administered to rats via injection 1 to 5 times a day for 3-10 days. Attempt is then made to initiate polyarthritis in these rats by injecting about 300-1000 ug of heat-killed Mycobacterium tuberculosis suspended in squalene into the tail vein of rats. Activity of disease is assessed by measuring the swelling of all four paws with a micrometer screw gauge. An overall arthritis score is independently assessed. The activity of the various amounts of CGP is compared to that of the control anti-inflammatory agent and that of the placebo.

A prophylactically effective amount of a glycerophosphate salt will reduce the overall arthritis score of the rats, or the probability for the onset of polyarthritis, by at least about 20%, for example, by at least about 30%, about 40%, about 50%, about 60%, about 70%, about 80%, about 90%, or about 100%.

### EXAMPLE 3

### Evaluating the Side Adverse Effect of Calcium Glycerophosphate on Gastrointestinal Tract in an Animal Model

This example depicts a gastroprotection assay to evaluate the side adverse effect, if any, of calcium glycerophosphate (CGP) on gastrointestinal tract in rats. Methods similar to that exemplified herein, however, are equally applicable to determining the side adverse effect of any glycerophosphate salt on gastrointestinal tract in any animals. Suitable assays for gastroprotection include, inter alia, those described by Rainsford and Whitehouse J. Pharm. Pharmacol. 44:476-482 (1992).

Rats are injected with an arthritogenic adjuvant or 0.1 ml oleyl alcohol 5 days prior to the gastroprotection assays. The gastrointestinal mucosa of rats is thus pre-sensitized by the development of arthritis or oleyl alcohol-induced inflammation in the rats. The animals are then fasted for 16 hours and given, orally or parenterally, a standard dose of 50 mg/kg ibuprofen free acid followed by various amounts of CGP, e.g., about 0.1 gram to about 3.0 grams per administration, or a placebo. The mean number of gastric lesions elicited by the ibuprofen is then measured from rats administered with the various amounts of CGP, and compared to that measured from rats administered with the placebo.

The therapeutically effective amount or the prophylactically effective amount of CGP should cause no or minimum level of additional gastric lesions to the rats.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

We claim:

1. A method of preventing or treating an inflammatory condition in a subject comprising administering to the subject an effective amount of a glycerophosphate salt, to interdict, prevent, palliate, alleviate, or delay the onset of the inflammatory condition in the subject, provided that the inflammatory condition is associated with one or more diseases other than those selected from the group consisting of irritable bowel syndrome, interstitial cystitis, hemorrhagic cystitis, inflammatory bowel disease and fibromyalgia.

2. The method of claim 1, wherein the inflammatory condition is manifested by one or more symptoms selected from the group consisting of pain, heat, redness, swelling, and a loss of function.

3. The method of claim 1, wherein the inflammatory condition is associated with a disease or a condition selected from the group consisting of rheumatoid arthritis, osteoarthritis, arthropathy, ankylosing spondylitis, psoriatic arthritis, reactive arthritis, gout, dysmenorrhoea, metastatic bone pain, headache, migraine, postoperative pain, mild-to-moderate pain due to inflammation and tissue injury, renal colic, appendicitis, gastritis, otitis, pancreatitis, laryngitis, asthma, chronic pelvic pain syndrome, chronic abacterial prostatitis, insult, trauma, an allergic response, an allergic reaction, mucositis, vulvar vestibulitis, a condition involving a mast cell mediator, and combinations thereof.

4. The method of claim 3, wherein the arthropathy is selected from the group consisting of reactive arthropathy, enteropathic arthropathy, crystal arthropathy, diabetic arthropathy, neuropathic arthropathy, and combinations thereof.

5. The method of claim 3, wherein the mast cell mediator is selected from the group consisting of a histamine, a cytokine, and a proteolytic enzyme.

6. The method of claim 1, wherein the inflammatory condition is associated with an inflammation selected from the group consisting of a brain inflammation, a meningeal inflammation, a skin inflammation, an inflammatory arthritis, a cardiopulmonary inflammation, and a gastrointestinal inflammation.

7. The method of claim 1, wherein the inflammatory condition is at least one of an internal and external inflammatory conditions.

8. The method of claim 1, wherein the glycerophosphate salt is selected from the group consisting of calcium glycerophosphate, magnesium glycerophosphate, zinc glycerophosphate, manganese glycerophosphate, lithium glycerophosphate, cupric glycerophosphate, ferric glycerophosphate, quinine glycerophosphate, glycerophosphate disodium, glycerophosphate dipotassium, glycerophosphate barium, glycerophosphate strontium, and combinations thereof.

9. The method of claim 8, wherein the glycerophosphate salt is calcium glycerophosphate.

10. The method of claim 9, wherein the inflammatory condition is mucositis, and the calcium glycerophosphate is administered topically to a mucous membrane.

11. The method of claim 9, wherein the inflammatory condition is vulvar vestibulitis, and the calcium glycerophosphate is administered topically to a vulvar surface.

12. The method of claim 9, wherein the inflammatory condition is pain.

13. The method of claim 1, wherein the subject is a human subject.

14. The method of claim 1, wherein the glycerophosphate salt is administered to the subject by one or more routes of administration selected from the group consisting of nasal administration; oral administration; inhalation administration; subcutaneous administration; transdermal administration; intravenous administration; topical administration; buccal administration; intraperitoneal administration; intraocular administration; peri-ocular administration; intramuscular administration; implantation administration; infusion, intravesical instillation; and central venous administration.

15. The method of claim 1, wherein the glycerophosphate salt is administered to the subject in a composition of a form selected from the group consisting of a cream, an ointment, a gel, a tablet, a capsule, a pill, a caplet, a granule, a powder, a syrup, a solution, a syrup, an elixir, an emulsion, a suppository a suspension, a spray, drops, and sustained release formulations.

16. The method of claim 1, wherein the effective amount of the glycerophosphate salt is about 0.1 gram to about 3.0 grams per administration.

17. The method of claim 1, wherein the glycerophosphate salt is administered to the subject in combination with an agent selected from the group consisting of a second anti-inflammatory agent, an anti-histamine agent, pentosan polysulfate (PPS), an anti-infective agent, a heparin-like macromolecular carbohydrate derivative, and combinations thereof.

18. The method of claim 17, wherein the second anti-inflammatory agent is selected from the group consisting of a second glycerophosphate salt, a corticosteroid, and a non-steroidal anti-inflammatory drug.

19. The method of claim 18, wherein the non-steroidal anti-inflammatory drug is aspirin.

20. The method of claim 17, wherein the anti-infective agent is selected from the group consisting of an antibiotic drug, an alcohol, and povidone.
